(19) United States
(12) Patent Application Publication
(10) Pub. No.: US 2022/0304632 A1
(43) Pub. Date: Sep. 29, 2022

(54) SEIZURE EARLY-WARNING METHOD AND SYSTEM

(71) Applicant: SENSOMICS, INC., Burlingame, CA (US)

(72) Inventors: Lichen ZHU, Burlingame, CA (US); Sixing LIU, Burlingame, CA (US); Jingjing LI, Burlingame, CA (US); Zhaolei ZHANG, Burlingame, CA (US)

(21) Appl. No.: 17/838,288

(22) Filed: Jun. 13, 2022

Related U.S. Application Data
(63) Continuation of application No. PCT/CN2019/126474, filed on Dec. 19, 2019.

(51) Int. Cl. A61B 5/00; G16H 50/20; G16H 10/60; G16H 50/70; G16H 40/63; A61B 5/11; A61B 5/0205; A61B 5/0531

(52) U.S. Cl. CPC A61B 5/746; G16H 50/20; G16H 10/60; G16H 50/70; G16H 40/63; A61B 5/4094; A61B 5/1122; A61B 5/02055; A61B 5/6801; A61B 5/7267; A61B 5/7275; A61B 5/0531; A61B 5/02405

(57) ABSTRACT

A seizure early-warning method and a system thereof, relating to a technical field of artificial intelligence. The method includes: acquiring health data of a user (S110); extracting first feature parameters from the acquired health data (S120); inputting the extracted first feature parameters into a preset seizure probability estimation model, so as to obtain a seizure probability (S130), wherein the seizure probability estimation model being generated by training a classification model by means of historical health data of a plurality of patients, and the historical health data of the patients including health data of the patients before the seizure; and determining, according to the seizure probability, whether to issue a seizure early-warning notification (S140). The system is a system configured to execute the method. The described technical solution can implement seizure early-warning.

### CROSS-REFERENCE TO RELATED APPLICATION

This application is a continuation of international application No. PCT/CN2019/126474 filed on Dec. 19, 2019, the content of which is hereby incorporated by reference in its entirety.

### TECHNICAL FIELD

The present disclosure generally relates to a technical field of artificial intelligence, and in particular, to a seizure early-warning method and a system thereof.

### BACKGROUND

A seizure is an acute, recurrent, and paroxysmal disorder of brain function caused by excessive neuronal discharge in the brain, which manifests as consciousness, motor, vegetative nerve and mental disorders. The seizure usually occurs suddenly, and if a patient cannot be rescued in time when the seizure occurs, a consequence will be severe and even life-threatening. A conventional solution for detecting seizure is based on an implantation of a brainwave detecting device into the human body and an analysis of a brainwave signal by the brainwave detection device to detect the seizure. However, this solution can only detect whether the seizure has occurred and usually sends an alert after the seizure, rather than early-warning of the seizure.

### SUMMARY

It is desired to provide a seizure early-warning method and a system thereof.

The seizure early-warning method includes: acquiring health data of a user; extracting first feature parameters from the acquired health data, inputting the extracted first feature parameters into a preset seizure probability estimation model, so as to obtain a seizure probability, and determining, according to the seizure probability, whether to send a seizure early-warning notification. The seizure probability estimation model is generated by training a classification model by means of historical health data of a plurality of patients, and the historical health data of the plurality of patients includes health data of the plurality of patients before the seizure.

In an embodiment, the classification model includes a dichotomous model, the historical health data of the plurality of patients further includes health data of the plurality of patients upon a condition that the plurality of patients are in a normal state, and the seizure probability estimation model is established by: pre-processing the health data of the plurality of patients before the seizure and the health data of the plurality of patients upon a condition that the plurality of patients are in the normal state respectively, extracting second feature parameters from the pre-processed health data of the plurality of patients before the seizure, extracting third feature parameters from the pre-processed health data of the plurality of patients in the normal state, and taking the patient being in a seizure state as a first dependent variable and the patient being in a normal state as a second dependent variable and training the dichotomous model based on the second feature parameters and the third feature parameters to obtain the seizure probability estimation model.

In an embodiment, the acquiring health data of the user further includes acquiring health data of the user during a first preset time. The historical health data of the plurality of patients includes health data of the plurality of patients during the first preset time before the seizure.

In an embodiment, the determining, according to the seizure probability, whether to send the seizure early-warning notification further includes: determining whether the seizure probability is greater than a preset threshold, and in response to determining that the seizure probability is greater than the preset threshold, sending the seizure early-warning notification.

In an embodiment, the extracting first feature parameters from the acquired health data further includes: pre-processing the acquired health data, and extracting the first feature parameters from the pre-processed health data.

In an embodiment, the health data of the user includes physiological parameters of the user, and the pre-processing the acquired health data further includes subtracting corresponding baseline correction values from the physiological parameters of the user. The baseline correction values are obtained by subtracting preset target physiological parameters from pre-obtained physiological parameters of the user at rest.

In an embodiment, the physiological parameters of the user include a heart rate, a skin temperature, and a skin resistance of the user.

In an embodiment, the health data of the user further includes movement parameters of the user, which include an angular velocity and an acceleration collected by a wearable device carried by the user.

In an embodiment, the first feature parameters include feature parameters of the heart rate, and the feature parameters of the heart rate are acquired by calculating a ventricular beat spacing and heart rate variability of the user based on the heart rate of the user.

In an embodiment, the first feature parameters include a first motion feature parameter, and the first motion feature parameter is acquired by: determining step count of the user as the first motion feature parameter according to the angular velocity and the acceleration.

In an embodiment, the first feature parameters include a second motion feature parameter, and the second motion feature parameter is acquired by: determining a moving distance of the user as the second motion feature parameter according to the angular velocity and the acceleration.

In an embodiment, the first feature parameters include a third motion feature parameter, and the third motion feature parameter is acquired by: determining a trajectory of the user as the third motion feature parameter according to the angular velocity and the acceleration.

In an embodiment, the health data of the user further includes identification information of the user, which includes an age and a gender of the user.

A seizure early-warning system provided includes a memory and a processor. The memory stores a computer program, and the processor is configured to execute the computer program to implement the above seizure early-warning method.

### BRIEF DESCRIPTION OF THE DRAWINGS

To describe and illustrate embodiments and/or examples of the present disclosure made public here better, reference may be made to one or more of the figures. The additional details or embodiments used to describe the figures should not be construed as limiting the scope of any of the present disclosure, the embodiments and/or examples currently described, and the best model of the present disclosure as currently understood.

FIG. 1 is a flowchart diagram of a seizure early-warning method according to a first embodiment of the present disclosure.

FIG. 2 is a block diagram of a structure of a seizure early-warning device according to a second embodiment of the present disclosure.

FIG. 3 is a block diagram of a structure of a seizure early-warning system according to a third embodiment of the present disclosure.

In the figures, 210 represents an acquiring module; 220 represents an extracting module; 230 represents a probability estimation module; 240 represents a determining module; 310 represents a processor; 320 represents a computer program; 330 represents a memory.

### DETAILED DESCRIPTION OF THE EMBODIMENT

The technical solutions in the embodiments of the present disclosure are clearly and completely described in the following with reference to the accompanying drawings in the embodiments of the present disclosure. It is obvious that the described embodiments are only a part of the embodiments, but not all of the embodiments. All other embodiments obtained by those skilled in the art based on the embodiments of the present disclosure without making creative labor are the scope of the present disclosure.

Unless otherwise defined, all technical and scientific terms used herein have the same meaning as a skilled person in the art would understand. The terminology used in the description of the present disclosure is for the purpose of describing particular embodiments and is not intended to limit the disclosure. The term “or/and” as used herein includes any and all combinations of one or more of the associated listed items.

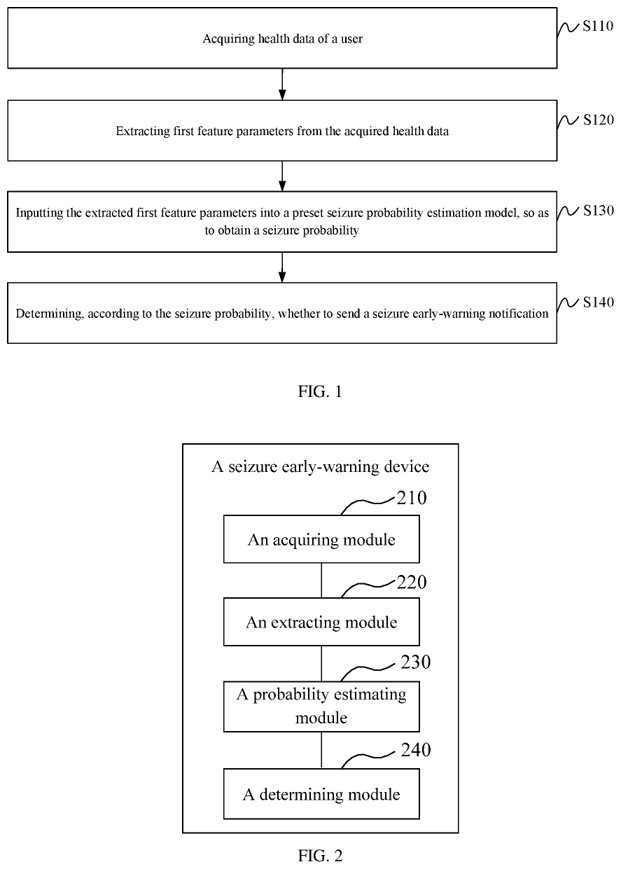

FIG. 1 is a flowchart diagram of a seizure early-warning method according to a first embodiment of the present disclosure. The seizure early-warning method provided in the first embodiment can be performed by a device with a processer such as a server, a cellphone, a laptop or a tablet. In the first embodiment, the server is described as an example.

In the first embodiment, the server can establish a data connection with at least one external device. The external device can be a cellphone or a tablet, etc. Communication means between the external device and the server for data connection is not limited. For example, the communication means can be a USB (Universal Serial Bus) connection, a LAN (Local Area Network), Internet, a Bluetooth, a WI-FI (Wireless local Area Network) or a Zigbee (Purple Bee Protocol), etc. In the first embodiment, the external device is described as a cellphone.

Furthermore, upon a condition that the server interacts with at least one cellphone, the cellphone acts as a client. In general, the cellphone can be one or more.

Furthermore, the cellphone can establish a data connection with at least one data collection device. The data collection device can be a device with a sensor such as a wearing device. The wearing device can include, but is not limited to, a bracelet, a patch, a watch, or an apparel, etc. Communication means between the cellphone and the data collection device for data connection is not limited. For example, the communication means can be a USB (Universal Serial Bus) connection, a LAN (Local Area Network), Internet, a Bluetooth, a WI-FI (Wireless local Area Network) or a Zigbee (Purple Bee Protocol), etc.

Specifically, referring to FIG. 1, the seizure early-warning method provided in the first embodiment can include the following steps.

At step 110, health data of a user can be acquired.

Specifically, the health data of the user can include physiological parameters of the user. The physiological parameters of the user can include a heart rate, a skin temperature, and a skin resistance of the user. Specifically, the cellphone can acquire the physiological parameters of the user by the wearing device. Compared with only acquiring the movement parameters of the user, acquiring physiological parameters of the user can enable a detection of seizure to be different from a detection of a convulsive behavior, which can realize the seizure early-warning more accurately.

Furthermore, the health data of the user can further include movement parameters of the user. The movement parameters of the user can include an angular velocity and an acceleration collected by a wearable device carried by the user. Specifically, the wearing device can include a gyroscope and an accelerometer. The wearing device can acquire the angular velocity by the gyroscope, and acquire the acceleration by the accelerometer. The cellphone can acquire the movement parameters of the user from the wearing device. Specifically, the gyroscope can include a triaxial gyroscope, and the accelerometer can include a triaxial accelerometer. Accuracy of seizure early-warning can be further improved by acquiring the movement parameters of the user, increasing influencing factors of seizure, and refining granularity of data analysis.

Furthermore, the health data of the user can further include identification information of the user. The identification information of the user can include an age and a gender of the user. Alternatively, the identification information of the user can be pre-stored in the wearing device or bound to the wearing device in other ways. For example, the identification information of the user can be bound in a client paired with the wearing device, the cellphone can acquire the identification information of the user by a data interface provided by the client. Accuracy of seizure early-warning can be further improved by acquiring the identification information of the user including the age and the gender of the user, increasing influencing factors of seizure, and refining granularity of data analysis.

The health data of the user can further include an identity of the wearing device of the user. The identity of the wearing device can be factory set or selected by the user from a list of a wearing device identities. Generally, a wearing device can correspond to a unique identity of the wearing device.

At step 120, first feature parameters can be extracted from the acquired health data.

Furthermore, the extracting first feature parameters from the acquired health data can further include: pre-processing the acquired health data, and extracting the first feature parameters from the pre-processed health data.

Furthermore, the pre-processing the acquired health data can include: subtracting corresponding baseline correction values from the physiological parameters of the user. The baseline correction values are obtained by subtracting preset target physiological parameters from pre-obtained physiological parameters of the user at rest.

Furthermore, the pre-processing the acquired health data can further include normalizing, smoothing, and unique thermal coding the acquired health data.

Furthermore, the extracting the first feature parameters from the pre-processed health data can further include: statistically extracting the pre-processed health data respectively to obtain the first feature parameters.

Alternatively, the first feature parameters can include feature parameters of a heart rate. The statistically extracting the pre-processed health data respectively to obtain the first feature parameters can include: calculating a RR interval (a ventricular beat spacing) and heart rate variability of the user based on the heart rate of the user to obtain the feature parameters of the heart rate. The feature parameters of the heart rate can be easier to obtain compared to feature parameters of brain wave and can reduce difficulty of seizure early-warning.

Alternatively, the first feature parameters can include a first motion feature parameter. The first motion feature parameter can be acquired by: determining step count of the user as the first motion feature parameter according to the angular velocity and the acceleration.

Alternatively, the first feature parameters can include a second motion feature parameter. The second motion feature parameter can be acquired by: determining a moving distance of the user as the second motion feature parameter according to the angular velocity and the acceleration.

Alternatively, the first feature parameters can include a third motion feature parameter. The third motion feature parameter can be acquired by: determining a trajectory of the user as the third motion feature parameter according to the angular velocity and the acceleration.

At step 130, the extracted first feature parameters can be input into a preset seizure probability estimation model, so as to obtain a seizure probability. The seizure probability estimation model can be generated by training a classification model by means of historical health data of a plurality of patients, and the historical health data of the plurality of patients can include health data of the plurality of patients before the seizure.

The classification model can include, but is not limited to, a Logistic Regression, a support vector machine, a Multi-layer Perception, a K Nearest Neighbor, or a random forest.

The seizure probability estimation model can be generated by training the classification model with the historical health data of the plurality of patients to achieve machine learning, and the seizure probability estimation model can be used to estimate the seizure probability of the user to improve accuracy of the probability estimation. Since the historical health data of the plurality of patients can include health data of the plurality of patients before the seizure, the seizure probability of the user can be predicted by inputting the health data of the user to the seizure probability estimation model, thus achieving a purpose of seizure early-warning.

Furthermore, the classification model can include a dichotomous model, the historical health data of the plurality of patients can further include health data of the plurality of patients upon a condition that the plurality of patients are in a normal state.

The seizure probability estimation model can be established by: pre-processing the health data of the plurality of patients before the seizure and the health data of the plurality of patients upon a condition that the plurality of patients are in the normal state respectively, extracting second feature parameters from the pre-processed health data of the plurality of patients before the seizure, extracting third feature parameters from the pre-processed health data of the plurality of patients in the normal state, and taking the patient being in a seizure state as a first dependent variable and the patient being in a normal state as a second dependent variable and training the dichotomous model based on the second feature parameters and the third feature parameters to obtain the seizure probability estimation model.

At step 140, it is determined according to the seizure probability that whether to send a seizure early-warning notification.

Furthermore, the determining whether to send the seizure early-warning notification according to the seizure probability can include determining whether the seizure probability is greater than a preset threshold, and in response to determining that the seizure probability is greater than the preset threshold, sending the seizure early-warning notification.

For example, the preset threshold is denoted as 0.5, and upon a condition that the seizure probability is greater than 0.5, the user can be considered likely to have a seizure and the seizure early-warning notification can be sent. It should be noted that 0.5 is only an exemplary illustration, and examples of the present disclosure are not limited.

Furthermore, the acquiring health data of the user can include acquiring health data of the user during a first preset time. The historical health data of the plurality of patients can include health data of the plurality of patients during the first preset time before the seizure.

For example, the health data of the user during 5 minutes before the current moment can be acquired. The historical health data of the plurality of patients can include the health data of the plurality of patients during 5 minutes before the seizure.

A reliability of the model can be improved by using the health data of the plurality of the patients during the first preset time before the seizure, as opposed to obtaining data at a certain moment. The health data of the user during the first preset time can be processed, and the seizure probability of the user after the first preset time can be estimated by the seizure probability estimation model, which can in turn enable seizure early-warning.

Alternatively, a seizure early-warning application can be installed in the cellphone and/or the server. The seizure early-warning application in the cellphone can be configured to transmit the health data of the user to the server. The seizure early-warning application in the server can be configured to receive the health data of the user from the cellphone, generate an analysis result of seizure early-warning based on the health data of the user, and transmit the generated analysis result of seizure early-warning to the cellphone.

Furthermore, the seizure early-warning application in the cellphone can further be configured to receive the analysis result of seizure early-warning from the server, receive a record of added seizures, and transmit the record of added seizures to the server at regular intervals.

Furthermore, the seizure early-warning application in the server can further be configured to receive the record of added seizures from the cellphone at regular intervals, and update the seizure probability estimation model according to the record of added seizures, further improving accuracy of the seizure probability estimation.

Furthermore, the seizure early-warning application in the cellphone can further be configured to determine whether the cellphone is disconnected from the server. Upon a condition that the cellphone is disconnected from the server, the seizure early-warning application can input the health data of the user into the seizure probability estimation model pre-loaded from the server to obtain the seizure probability of the user, and determine whether to send the seizure early-warning notification according to the seizure probability.

Furthermore, the seizure early-warning notification can include alarms, beeps, text messages, preset messages, etc.

FIG. 2 is a block diagram of a structure of a seizure early-warning device according to a second embodiment of the present disclosure. In the present embodiment, the seizure early-warning device can include an acquiring module 210, an extracting module 220, a probability estimation module 230, and a determining module 240.

The acquiring module 210 is configured to acquire health data of a user.

The extracting module 220 is configured to extract first feature parameters from the acquired health data.

The probability estimation module 230 is configured to input the extracted first feature parameters into a preset seizure probability estimation model, so as to obtain a seizure probability. The seizure probability estimation model can be generated by training a classification model by means of historical health data of a plurality of patients, and the historical health data of the plurality of patients can include health data of the plurality of patients before the seizure.

The determining module 240 is configured to determine, according to the seizure probability, whether to send a seizure early-warning notification.

The seizure probability estimation model can be generated by training the classification model with the historical health data of the plurality of patients to achieve machine learning, and the seizure probability estimation model can be used to estimate the seizure probability of the user to improve accuracy of the probability estimation. Since the historical health data of the plurality of patients can include health data of the plurality of patients before the seizure, the seizure probability of the user can be predicted by inputting the health data of the user to the seizure probability estimation model, thus achieving a purpose of seizure early-warning.

Furthermore, the classification model can include a dichotomous model, the historical health data of the plurality of patients can further include health data of the plurality of patients upon a condition that the plurality of patients are in a normal state.

An establishing module of the seizure probability estimation model can include a first pre-processing unit, a first feature extraction unit, a second feature extraction unit, and a training unit.

The first pre-processing unit is configured to pre-process the health data of the plurality of patients before the seizure and the health data of the plurality of patients upon a condition that the plurality of patients are in the normal state respectively.

The first feature extraction unit is configured to extract second feature parameters from the pre-processed health data of the plurality of patients before the seizure.

The second feature extraction unit is configured to extract third feature parameters from the pre-processed health data of the plurality of patients in the normal state.

The training unit is configured to take the patient being in a seizure state as a first dependent variable and the patient being in a normal state as a second dependent variable, and train the dichotomous model based on the second feature parameters and the third feature parameters to obtain the seizure probability estimation model.

Furthermore, the acquiring module 210 can include a health data acquiring unit configured to acquire health data of the user during a first preset time. The historical health data of the plurality of patients can include health data of the plurality of patients during the first preset time before the seizure.

Furthermore, the determining module 240 can include a determining unit and a first responding unit.

The determining unit is configured to determine whether the seizure probability is greater than a preset threshold.

The first responding unit is configured to send a seizure early-warning notification in response to determining that the seizure probability is greater than the preset threshold.

In an embodiment, the determining module 240 can further include a second responding unit.

The second responding unit is configured to re-execute the seizure early-warning method in response to determining that the seizure probability is less than or equal to the preset threshold.

In an embodiment, the extracting module 220 can include a second pre-processing unit and a third feature extraction unit.

The second pre-processing unit is configured to pre-process the acquired health data.

The third feature extraction unit is configured to extract the first feature parameter from the pre-processed health data.

In an embodiment, the health data of the user can include physiological parameters of the user. The second pre-processing unit can include a baseline correction sub-unit configured to subtract corresponding baseline correction values from the physiological parameters of the user. The baseline correction values are obtained by subtracting preset target physiological parameters from pre-obtained physiological parameters of the user at rest.

In an embodiment, the physiological parameters of the user can include a heart rate, a skin temperature, and a skin resistance of the user.

In an embodiment, the health data of the user can further include movement parameters of the user. The movement parameters of the user can include step count of the user.

In an embodiment, the health data of the user can further include identification information of the user. The identification information of the user can include an age and a gender of the user.

The above seizure early-warning device is configured to perform the seizure early-warning method provided in any one of the above embodiments with corresponding functionality and beneficial effects.

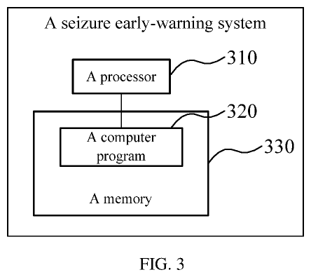

FIG. 3 is a block diagram of a structure of a seizure early-warning system according to a third embodiment of the present disclosure. In the present embodiment, the seizure early-warning system can include a processor 310 and a memory 330. The memory 330 can store a computer program 320, and the processor 310 is configured to execute the computer program 320 to implement the seizure early-warning method in any one of the above embodiments.

The seizure early-warning system can further include an input and output device, a network access device, a bus, etc.

The processor can be a Central Processing Unit (CPU), and can also be other general processor, a Digital Signal Processor (DSP), an Application Specific Integrated Circuit (ASIC), a Field-Programmable Gate Array (FPGA), other programmable logic device, a discrete gate or a transistor logic device, discrete hardware components, etc. The general processor can be a microprocessor or any conventional processor, etc. The processor is a control center of the seizure early-warning system and is connected with various parts of the entire seizure early-warning system through various interfaces and wirings.

The memory can mainly include a storage program area and a storage data area. The storage program area can store an operating system and applications required for at least one function (e.g., sound play function, image play function, etc.), etc. The storage data area can store data created according to the use of the cellphone (e.g., audio data, phone book, etc.), etc. In addition, the memory can include a high-speed random access memory, and further include non-volatile memory, such as a hard disk, an internal memory, a plug-in hard disk, a Smart Media Card (SMC), a Secure Digital (SD) card, a Flash Card, at least one disk memory device, a flash memory device, or other volatile solid state memory device.

All or part of the processes in the methods of the above embodiments can be performed by means of a computer program to instruct the relevant hardware to do so. The computer program may be stored in a computer readable storage medium. When the computer program is executed, the device in which the computer readable storage medium is located can be controlled to implement the seizure early-warning method as described in any of the above embodiment. The computer program can include computer program codes in a source code form, an object code form, an executable file, or some intermediate form, etc. The computer readable medium can include: any entity or device capable of carrying the computer program codes, a recording medium, a USB flash drive, a removable hard drive, a disk, an optical disk, a computer memory, a read-only memory (ROM), a random access memory (RAM), an electrical carrier signal, a telecommunication signal, and software distribution media, etc.

The technical features of the above-described embodiments may be combined in any combination. For the sake of brevity of description, not all possible combinations of the technical features in the above embodiments are described. However, as long as there is no contradiction between the combinations of these technical features, all should be considered as within the scope of this disclosure.

The above-described embodiments are merely illustrative of several embodiments of the present disclosure, and the description thereof is relatively specific and detailed, but is not to be construed as limiting the scope of the disclosure. It should be noted that a number of variations and modifications may be made by those skilled in the art without departing from the spirit and scope of the disclosure. Therefore, the scope of the disclosure should be determined by the appended claims.

We claim:

1. A seizure early-warning method comprising:
acquiring health data of a user;
extracting first feature parameters from the acquired health data;
inputting the extracted first feature parameters into a preset seizure probability estimation model, so as to obtain a seizure probability, wherein the seizure probability estimation model is generated by training a classification model by means of historical health data of a plurality of patients, and the historical health data of the plurality of patients comprises health data of the plurality of patients before the seizure; and
determining, according to the seizure probability, whether to send a seizure early-warning notification.

2. The method of claim 1, wherein the classification model comprises a dichotomous model, the historical health data of the plurality of patients further comprises health data of the plurality of patients upon a condition that the plurality of patients are in a normal state, and
the seizure probability estimation model is established by:
pre-processing the health data of the plurality of patients before the seizure and the health data of the plurality of patients upon a condition that the plurality of patients are in the normal state respectively;
extracting second feature parameters from the pre-processed health data of the plurality of patients before the seizure;
extracting third feature parameters from the pre-processed health data of the plurality of patients in the normal state; and
taking the patient being in a seizure state as a first dependent variable and the patient being in a normal state as a second dependent variable and training the dichotomous model based on the second feature parameters and the third feature parameters to obtain the seizure probability estimation model.

3. The method of claim 1, wherein the acquiring health data of the user further comprises:
acquiring health data of the user during a first preset time;
wherein the historical health data of the plurality of patients comprises health data of the plurality of patients during the first preset time before the seizure.

4. The method of claim 1, wherein the determining, according to the seizure probability, whether to send the seizure early-warning notification further comprises:
determining whether the seizure probability is greater than a preset threshold; and
in response to determining that the seizure probability is greater than the preset threshold, sending the seizure early-warning notification.

5. The method of claim 1, wherein the extracting first feature parameters from the acquired health data further comprises:
pre-processing the acquired health data; and
extracting the first feature parameters from the pre-processed health data.

6. The method of claim 5, wherein the health data of the user comprises physiological parameters of the user, and
the pre-processing the acquired health data further comprises:
subtracting corresponding baseline correction values from the physiological parameters of the user, wherein the baseline correction values are obtained by subtracting preset target physiological parameters from pre-obtained physiological parameters of the user at rest.

7. The method of claim 6, wherein the physiological parameters of the user comprise a heart rate, a skin temperature, and a skin resistance of the user.

8. The method of claim 7, wherein the health data of the user further comprises movement parameters of the user, which comprise an angular velocity and an acceleration collected by a wearable device carried by the user.

9. The method of claim 7, wherein the first feature parameters comprise feature parameters of the heart rate, and the feature parameters of the heart rate are acquired by calculating a ventricular beat spacing and heart rate variability of the user based on the heart rate of the user.

10. The method of claim 8, wherein the first feature parameters comprise a first motion feature parameter, and the first motion feature parameter is acquired by:
determining step count of the user as the first motion feature parameter according to the angular velocity and the acceleration.

11. The method of claim 8, wherein the first feature parameters comprise a second motion feature parameter, and the second motion feature parameter is acquired by:
determining a moving distance of the user as the second motion feature parameter according to the angular velocity and the acceleration.

12. The method of claim 8, wherein the first feature parameters comprise a third motion feature parameter, and the third motion feature parameter is acquired by:
determining a trajectory of the user as the third motion feature parameter according to the angular velocity and the acceleration.

13. The method of claim 8, wherein the health data of the user further comprises identification information of the user, which comprises an age and a gender of the user.

14. A seizure early-warning system comprising a memory and a processor, wherein the memory stores a computer program, and the processor is configured to execute the computer program to implement a seizure early-warning method comprising:
acquiring health data of a user;
extracting first feature parameters from the acquired health data;
inputting the extracted first feature parameters into a preset seizure probability estimation model, so as to obtain a seizure probability, wherein the seizure probability estimation model is generated by training a classification model by means of historical health data of a plurality of patients, and the historical health data of the plurality of patients comprises health data of the plurality of patients before the seizure; and
determining, according to the seizure probability, whether to send a seizure early-warning notification.

15. The system of claim 14, wherein the classification model comprises a dichotomous model, the historical health data of the plurality of patients further comprises health data of the plurality of patients upon a condition that the plurality of patients are in a normal state, and
the processor is further configured to execute the computer program to establish the seizure probability estimation model as follows:
pre-processing the health data of the plurality of patients before the seizure and the health data of the plurality of patients upon a condition that the plurality of patients are in the normal state respectively;
extracting second feature parameters from the pre-processed health data of the plurality of patients before the seizure;
extracting third feature parameters from the pre-processed health data of the plurality of patients in the normal state; and
taking the patient being in a seizure state as a first dependent variable and the patient being in a normal state as a second dependent variable and training the dichotomous model based on the second feature parameters and the third feature parameters to obtain the seizure probability estimation model.

16. The system of claim 14, wherein the acquiring health data of the user further comprises:
acquiring health data of the user during a first preset time;
wherein the historical health data of the plurality of patients comprises health data of the plurality of patients during the first preset time before the seizure.

17. The system of claim 14, wherein the determining, according to the seizure probability, whether to send the seizure early-warning notification further comprises:
determining whether the seizure probability is greater than a preset threshold; and
in response to determining that the seizure probability is greater than the preset threshold, sending the seizure early-warning notification.

18. The system of claim 14, wherein the extracting first feature parameters from the acquired health data further comprises:
pre-processing the acquired health data; and
extracting the first feature parameters from the pre-processed health data.

19. The system of claim 18, wherein the health data of the user comprises physiological parameters of the user, and
the pre-processing the acquired health data further comprises:
subtracting corresponding baseline correction values from the physiological parameters of the user, wherein the baseline correction values are obtained by subtracting preset target physiological parameters from pre-obtained physiological parameters of the user at rest.

20. The system of claim 19, wherein the physiological parameters of the user comprise a heart rate, a skin temperature, and a skin resistance of the user.
